(19) United States
(12) Patent Application Publication
(10) Pub. No.: US 2018/0055708 A1
(43) Pub. Date: Mar. 1, 2018

(54) Upper Extremity Positioner

(71) Applicant: Robert S. Hatch, Ormonk Beach, FL (US)

(72) Inventor: Robert S. Hatch, Ormonk Beach, FL (US)

(21) Appl. No.: 15/250,358

(22) Filed: Aug. 29, 2016

(51) Int. Cl. A61G 13/12; A61G 7/075; A61B 5/055; A61B 6/04

(52) U.S. Cl. CPC A61G 13/1235; A61G 13/124; A61B 6/0421; A61G 13/128; A61B 5/0555; A61G 7/075

(57) ABSTRACT

An apparatus and technique for supporting and securing a human upper extremity suitable for diagnostic and surgical procedures with the apparatus comprising a base plate coupled to moveable proximal and distal platforms. The distal platform supports the hand and wrist while pivoting about a ball and socket (universal) joint. The proximal platform is attached to a moveable support that variably elevates the upper extremity between the shoulder and the wrist (ie the distal platform) whilst travelling longitudinally along the base plate. These upper platforms attach to a common lower platform (ie the base plate) and function independently yet complimentarily to variably elevate and position the entire upper extremity above the base plate thereby achieving reproducible medical (or “user”) access to any discrete site from the shoulder to the digits. The proximal and distal platforms may also function as a bridge for a third upper platform converting an apparatus with two relatively short supportive surfaces to an apparatus having one relatively long supportive surface.

### FIELD OF INVENTION

This apparatus relates to a series of platforms and their attachments that reproduce and hold serviceable positions of the human upper extremity from the shoulder to the digits for evaluation and treatment purposes.

### BACKGROUND AND PRIOR ART

Patient positioning begins the series of events leading to medical diagnostic examinations and procedures for the upper extremity. Physicians and practitioners too often struggle with reproducible means of attaining ideal positioning. Small maladjustments in positioning can increase procedural time and decrease procedural accuracy. Without proven support apparatuses to guide patient positioning, practitioners are left to improvise set-ups using suboptimal props.

Advancing technologies such as musculoskeletal ultrasound are changing the method of extremity examination. New interventions derived from this technology demand precise upper extremity placement. To keep pace with new procedures, practitioners must evolve patient positioning techniques suitable for the required procedure. The ideal positioning system should offer these practitioners a simple-to-use apparatus with a facile and reproducible method for comfortably positioning a patient for review of anatomy.

Devices have been developed to aid in patient positioning of all body parts not to exclude the upper extremity. The majority of these devices are built either for the operating room setting, for anaesthetized patients, or for very specific anatomic sites such as “for the shoulder” or “for the hand” etc . . . . One still applicable comment describing the surgeons' dilemma for hand surgeries is excerpted from U.S. Pat. No. 4,858,903: “The operation on a hand . . . is generally performed by the operating surgeon with the aid of assistants. In the case of more serious operations, one assistant attempts to fix the hand in a suitable position, while another assists in the exposure following incision by using retractors”. This type of statement underscores the importance of a capable positioning device ie., to facilitate an exacting procedure by accurately holding an anatomical perspective thereby liberating the surgeon and assistants from the responsibility.

Practitioners performing upper extremity procedures outside the operating room (e.g., in a clinic) commonly find no such purposed positioning device. They rely on human assistance or the patients' ability to hold position without assistance or they use minimal support props such as with pillow-type cushions, gels, or towels. The problem with these inexact positioning methods is a combination of suboptimal positioning, overutilizaiton of human resources, and inability to precisely reproduce serviceable positions which leads to procedural inefficiencies. Furthermore, in the conscious patient, the lack of firm support may be confusing to the patient who must decide how best to cooperate with supports that do not provide adequate positional cues.

The rapidly growing area of invasive musculoskeletal ultrasound where a wide variety of diagnostic and short invasive procedures are performed on the upper extremity requires versatility of an upper extremity positioning system. Most of this work would be performed in the office setting with the remainder addressed in an operating room setting. As yet, no such purposed apparatus or system has been designed for general upper extremity positioning in both settings.

Procedures performed with greater efficiency by improved surgical ergonomics reduce medical cost and improve outcomes. For example, during an endoscopic carpal tunnel release where the wrist is held in a position of about 30 degrees of extension (dorsiflexion) most surgeons still use towels, gauze and manual help for hand and wrist positioning. Without firm support, as can be provided by an effective positioning apparatus, the surgery may be slowed by loss of positioning during the surgery, occupying an assistant that could be useful elsewhere, and unwanted movement of the poorly stabilized hand during the procedure.

The qualities of a useful upper extremity positioner such as portability, simplicity of mechanisms, quickness of positioning, sturdiness, sanitizing ease, ability to reproduce a variety of desired positions and patient comfort seem to be barriers for constructing an elective system. While the prior art has identified the need for improved upper extremity support systems there remains no generally accepted apparatus or described positioning technique or apparatus for general upper extremity positioning. Failure to remedy such a prevalent inadequacy exposes the difficulties in solving the problem of efficient patient positioning. Upon review of the prior art, one major reason for the paucity of ideas offered to manage upper extremity positioning is the dearth of research in the field of surgical ergonomics and patient positioning. To date, no patented upper extremity positioning device deconstructs movements of the human upper extremity by applied mechanical engineering. Another shortcoming of the prior attempts may be the high expense in developing complicated and cumbersome machines that address too narrow a clinical application or simply have too many moving parts to be used practically.

Examples of upper extremity positioning devices suboptimal for the clinical setting and having other notable surgical disadvantages are replete in the works of the prior art: The Pivoting Hand Table, U.S. Pat. No. 6,336,412-B2 is a flat table attaching to an operating room table that positions a portion of the upper extremity away from the patients' side. The table provides a surface for the upper extremity to rest upon but provides no customizeable positions for the arm otherwise. With this device the surgeon has to improvise or create additional support to perform many surgeries. The Pivoting Hand Table's simplistic concept is perhaps the most common type of operating room upper extremity table in use today, and may also be referred to as an “arm board” or a “hand table”.

The invention, Surgical prep block and surgical assist block, U.S. Pat. No. 4,681,309-A has utility for support just distal to the shoulder only and its surgical applications are not specifically discussed beyond being used for upper extremity pre-surgical preparations.

Invention, Hand Holding Apparatus for the Hand, U.S. Pat. No. 4,564,180, A (1986), describes a surgical support for the hand only. Unfortunately, positioning of the remainder of the upper extremity is not discussed but mechanics of the device obviate positioning of the arm, elbow and forearm during surgeries on the hand and wrist. The universal ball and socket joint is not eccentrically placed and lies near the center of the attached plate producing a nonphysiologic axis of rotation beneath the mid-palm. Small movements of the hand upon the tiltable table can produce elevation of the extremity causing “wrist to torso” length changes that necessitate repositioning of the entire upper extremity, but the invention does not describe the manner in which the remainder of the upper extremity is to be positioned once disturbed by a change in position of the wrist. A similar hand support of European design with application 85904587.4, Hand surgery tablet (filing date 1985), also uses a rotational swivel ball toward the middle of the apparatus, as demonstrated in a preferred embodiment, and suffers from the same mechanical awkwardness of inadvertent elbow motion produced by changes in hand and wrist positioning. These devices teach that small surgical hand tables may perform well within a narrow scope of fixed surgical positions exclusively for the hand, bat would likely require a more sophisticated apparatus to accommodate hand-wrist movements that also produced movements of the elbow.

Invention, Surgery Table, U.S. Pat. No. 4,082,257 A, supports the hand and forearm but demonstrates rigid restriction of the forearm and hand once placed on the apparatus. Customized surgical exposure opportunities are thus severely limited. Positioning of the remainder of the upper extremity or position of the patient is not addressed but the table appears to be favorably designed to provide support for the forearm and hand while the patient is lying in a supine position. Patient positions other than supine may be problematic for the curved design of the table surface. Additional support for the extremity above the elbow may be needed, but such discussion appears to be beyond the scope of the invention as written.

Invention Extremity Support Rack U.S. Pat. No. 7,156,820 B2 describes an apparatus that cradles an entire upper or lower extremity for settings where injury and/or trauma management may take place. The invention utilizes hingedly connected (see reference #106) upper and lower rails having cradles to support an extremity. This pivotal connection between the supporting rails provides obligatory and simultaneous movement of one rail when the other is moved. The support racks are adjustably elevated and positioned to fit injured limbs for purposes of applying rest, ice, compression and elevation. No other procedural uses are described. The apparatus is not designed for surgical purposes and otherwise seems equally useful for the upper and lower human extremities despite their vast differences in kinetic abilities.

Invention U.S. Pat. No. 4,858,903, Hand surgery operating table, describes a support for the forearm with same level, hand and wrist support. The device is preferably attached to an operating room table. The inventors posit that the hand and wrist are moveable presumably within a wide range, but these ranges are not listed nor are they pictorially represented. The device is suitable only for hand procedures and by which method the device maintains adequate control of the remainder of the upper extremity is not precisely defined.

The Upper Extremity Positioning System exemplifies design and methodological concepts developed from decades of operating room, office, musculoskeletal ultrasound experience, and exhaustive prototyping. The Upper Extremity Positioner will now be fully described.

The basic iterations of the Upper Extremity Positioner are constructed with a base plate, two platforms connected to the base plate independently, and with at least one of the platforms having the ability to change its position relative to the base plate. The base plate and the platforms are made from a stout material such as from the wood, plastic or metals family. The platforms are coupled to the base plate and aligned in such a way that an outstretched arm rests on both platform. The two platforms are elevated above the base plate by specialized attachments, such as a pillar or hinge, with variable positioning opportunities through adjustments of one or both platforms. Mobility of the platforms depends upon the geometry and the number of the connected moveable fasteners. Each moveable fastener effectively multiplies the spatial area covered by the mobile platform proportional to the number of moveable fasteners added.

This “basic iteration” is based on a conceptual framework for supporting and holding an upper extremity. The apparatus has two general sites of contact for an upper extremity, with one site being the hand and wrist and the second site being between the wrist and the shoulder. A third site is the patients shoulder and torso articulations (ie the shoulder girdle). This “three contact points” configuration allows the upper extremity to be optimally balanced as the shoulder serves as a universal joint styled support of the entire arm, while a distal platform supports the hand and wrist, and a second (proximal) platform receives the remaining weight of the arm ie, between the shoulder and the first platform. In this “basic iteration” the proximal platform is supported and moved by a hinge mechanism and the distal platform is supported and moved by a ball-and-socket joint fixed to a pillar.

The dual hinge mechanism performs two necessary functions for the proximal platform; the first (rotating) hinge couples directly to the proximal platform providing rotation with an axis of rotation perpendicular to the long axis of the base plate and a second (pivot) hinge translates the proximal platform “toward and away” from the wrist to accommodate varying arm lengths while elevating the proximal platform to an appropriate height to support the arm as it lies above or below the distal platform. This generalized proximal platform cooperates well with a distal platform having a stationary pillar with a ball and socket universal joint. While the distal platform works well in a fixed position (ie, without a ball and socket fastener), the ball-and-socket mechanism, which allows a wide range of motion for the distal platform, substantially improves overall performance of the apparatus. A distal platform constructed much like a tiltable hand table and a proximal platform having longitudinal and elevational and obligatory translational (long axis) movement abilities for positions along the upper extremity from the shoulder to the wrist are the preferred pillars and fasteners for a basic iteration of the Upper Extremity Positioner.

The advantage of having two elevated platforms instead of only one, as in Hand Holding Apparatus for the Hand, U.S. Pat. No. 4,564,180, is the proximal platform being adjustable as the position of the elbow needs to change. Elbow positional changes are the principle reason for the failure of small hand tables that only provide universal motion to the hand and wrist. On these single platforms as the hand-wrist is pivoted elevational changes of the platform (table) often necessitate movements of the elbow (and the segments proximal and distal to the elbow). The single platform does not accommodate changes in any segment other than the hand and the wrist. Due to failure of the single platform wrist/hand devices to provide a remedy for positional changes of the more proximal anatomy the practitioner is then burdened to improvise support for the changed position of the extremity proximal to the wrist and for any other such positional changes that follow.

From the “two elevated platforms” concept emerges the utility of at least a third platform called an intercalating platform. The intercalating platform forms a supportive bridge between the two platforms. If the distal platform is fitted with a universal joint (eg, ball and socket fastener) and the proximal platform is allowed to elevated and rotate by hinges then the supporting surfaces of the elevated platforms can be aligned in a uniplanar configuration with any elevational change in position of either platform. When the two supporting surfaces are uniplanar the flat intercalating platform (of the presented embodiment) rests atop both in the desired plane of elevation. In this way, the upper extremity positioner can be used as an “arm board” (as described above) ie to provide continuous support to whatever portion of the entire upper extremity is deemed useful.

The Upper Extremity Positioner System is easy to use and the practitioner is recommended to begin with a standardized positioning method, as will now be described. The practitioner must first decide the general position of the patient such as sitting, supine or in a lateral decubitus position (ie lying on ones side) or any acceptable position in between. This decision may be influenced by the doctors preference and the patients ability to attain the position. For example, cardiovascular comorbidity may cause a patient to be unable to lie completely supine (flat on their back) so the practitioner must then adapt the positioning technique to accommodate this limitation. The Upper Extremity Positioning System allows the practitioner to place the patient in a most comfortable position and then place the upper extremity in a suitable position for the planned procedure.

The standard Upper extremity Positioner method is to, first, place the patient in the generally desired position. The upper extremity Is then positioned for optimum review of the area of interest initially without The Upper Extremity Positioner System in place. The Upper Extremity Positioner apparatus is then placed on a moveable table, such as a common inexpensive hospital patient serving stand (which rolls and typically elevates and lowers to accommodate lying and sitting positions), and brought in beneath the positioned extremity. The hand is then placed on the hand distal support plate to achieve proper length relationships of the arm and the upper extremity positioned apparatus, and, finally, the proximal support plate would be elevated into position to complete support of the extremity. Various restraints, not to exclude the tension plate, can then be used to secure the hand, wrist, digits, and more proximal arm.

As previously stated the concept of the Upper Extremity Positioner System originated from recognizing the suboptimal abilities of towels, pillows, gauze and other props to adapt to a variety of patient circumstances. The need for such a device was most obvious in the clinical setting where there are fewer props and generally less manpower than provided by the typical operating room setting. Even short procedures such as injections as well as diagnostic ultrasound to all regions of the upper extremity were observed to have shortcomings not excluding loss of patient positioning during the procedure, inadequate elevation of a syringe from the arm, and patient confusion where the props failed to cue the conscious patient to properly hold still and not “drift” during the procedure. The Upper Extremity Positioner solves tire problem of reproducibly accessing large areas of upper extremity anatomy for procedural purposes.

While many unique devices have been properly invented there is no prior art demonstrating a positioner useful and practical for the entire upper extremity with recommended use in the office and the operating room setting. Similarly, there is no table configuration or positioning concept that has gained widespread popularity in the operating room or the clinical setting. The Upper Extremity Positioner system demonstrates a novel design to address many upper extremity procedures and through its design and utility presents a novel approach to upper extremity positioning thus establishing a clear and significant improvement over prior art, other hand/wrist support designs and improvised props in use currently.

### SUMMARY OF THE INVENTION

Embodiments of the Upper Extremity Positioner apply basic concepts for positioning an extremity. The first concept is that only two platforms are needed to provide sufficient support for most upper extremity procedures with one platform dedicated to the hand and at least a portion of the wrist, and a second platform free to move between the shoulder and the wrist. The hand platform (distal platform) preferably, but not absolutely, has a universal joint (ball and socket in this case) to maximize wrist motion. The wrist is preferably placed at the edge of the distal platform such that the forearm does not encroach onto the table and thereby limit wrist movement proportional to the degree of encroachment, and this position decreases obligatory movements of anatomy proximal to the wrist. Movements of the forearm and elbow that do occur as a necessary effect of wrist or forearm motion are accommodated by the easily adjusting proximal platform.

The two basic functions of the proximal support platform are first is to translate “toward and away” from the wrist to accommodate arm lengths, and, second, to elevate and support the arm proximal to the wrist to complete the “mid-arm” contact point. The basic function of the distal platform is to elevate the hand and wrist above the base support to allow movement of the hand without impingement against the base support, and to maximize positional opportunities of the hand-wrist by mimicking rotational wrist motion through a universal (ball-and-socket) joint. The two elevated support platforms can additionally be made uniplanar and then connected by a third support platform which converts the table to a traditional flat “arm board” support. Any joints, hinges or other mobile mechanisms added to the support platforms can substantially multiply positioning opportunities of the upper extremity.

While the above description contains many specificities, these should not be construed as limitations on the scope of any embodiment, but as exemplifications of various embodiments thereof. Many other ramifications and variations are possible within the teachings of the various embodiments. Thus it can be seen that although one embodiment of the upper extremity positioner is uniquely biased to the practice of its inventor there would be obvious modifications adapting the apparatus for other upper extremity work. Such useful adaptations are inherent within the concept of the apparatus as listed in the claims. For example, use of additional elevating and support planes, various suitable textured materials not to exclude radiolucent, lighter (or more portable) material, MRI compatible material, CT-scan compatible material or other imaging modality, additional attachments to any part of the assembly or additional support, telescoping poles in place of the hand support column and in place of the support(s) placed more proximally, adjustments in the size and weight of the apparatus, altering the distances between supports or inserting mechanisms and means for such, applying traction for additional stability and for trauma, battlefield or fracture management, varying the potential heights of the supports, placement of brackets and hardware to adapt the apparatus for use in the operating room, improvements in portability not to exclude a collapsible version with carrying case, attachments of devices to the apparatus not to exclude lighting sources, battery packs, lenses and microscopes, equipment and tool holders, modification in the size and shape of the support platforms, or nonmedical support uses to name but a few.

A primary purpose of the upper extremity positioner is as a versatile upper extremity apparatus for disciplined and reproducible positioning from the shoulder to the digits.

Another advantage of the upper extremity positioner is to offer a simple method for upper extremity placement thereby improving the reproducibility of desired positions of the shoulder, brachial region, elbow, forearm, wrist and hand.

Another purpose of the upper extremity positioner is to hold conscious patients still and comfortable while providing them with enough support so that cooperation with a given position on the apparatus feels intuitive.

Another purpose of the upper extremity positioner is for elevating the upper extremity above a support plane (table) through elevated platforms thus allowing surgical instrument clearance for such procedures.

Another purpose of the upper extremity positioner is teaching a three-point support concept with one point of stability being the body/torso (stabilizing the shoulder), a second point being the proximal support platform that stabilizes the arm between the shoulder and the wrist and a third point of stability being the hand/wrist supported by a distal platform.

Another advantage of the upper extremity positioner is as a concept that improves the efficiency of upper extremity office procedures and minimally invasive (keyhole) procedures.

Another advantage of the upper extremity positioner is exceptional portablity

Another advantage of the upper extremity positioner is the ability to convert from a 2 platform support system to a traditional arm board system while retaining the flexibility to vary the inclination of the extremity relative to the torso and the ground

Patient position is one of the first important steps when preparing for upper extremity procedures, surgeries and for some diagnostic procedures. The upper extremity positioner is a device designed to help the physician and the surgeon with properly approaching a selective region of interest and then securing the upper extremity with a reproducible and reliable technique. A three point system of contacts is employed as the most fundamental and basic embodiment with the body to support the shoulder (and the shoulder to allow free range of the entire upper extremity), the second support being at or near the elbow and the third support at the wrist and hand. The upper extremity can be additionally secured to the apparatus by notches, slots and holes about the periphery and interior of the support platforms. The relatively light weight mobile system can be used and adapted to any setting such as in clinic, the operating room or the battle field for quick positioning of an upper extremity for a designated procedure.

### BRIEF DESCRIPTION OF THE FIGURES

FIG. 1. The Upper Extremity Positioner at a side view. The distal end of the apparatus is to the left of the picture.

FIG. 2. The Upper Extremity Positioner at a side view with the distal end of the distal platform in a downward tilted position and the proximal platform fully reclined.

FIG. 3. The Upper Extremity Positioner at a side view with the distal platform in an upwardly tilted position and the proximal platform fully reclined.

FIG. 4. The Upper Extremity Positioner at a side view with the distal platform in a neutral position, the upper arm of the hinge nearly fully elevated, and the proximal platform in a tilted position.

FIG. 5. The upper extremity positioner holding an upper extremity in a surgical position.

FIG. 6. The distal platform holding a hand and wrist by restraining ties.

FIG. 7. A patient in a supine position with the upper extremity lying upon the Upper Extremity Positioner which has been placed on a table.

FIG. 8. Exploded view of the distal platform and its attachments to the pillar.

FIG. 9. The (dual) hinge assembly with the upper arm in an elevated position and the proximal platform in a tilted position.

FIG. 10. Exploded view of the (dual) hinge assembly (inferior-oblique perspective).

FIG. 11. A patient in a sitting position with the right arm lying on the Upper Extremity Positioner.

FIG. 12 The intercalating board with connecting pins.

FIG. 13 Exploded view of the intercalating board positioned so that the connecting pins align with holes at the proximal end of the distal platform.

FIG. 14 A patient in a supine position with the arm held at an angle by the Upper Extremity Positioner with the intercalating board in place.

FIG. 15 A patient lying in a right lateral decubitus position.

FIG. 16 A patient lying in a right lateral decubitus position with the left arm elevated away from the body.

FIG. 17. A patient lying in a right lateral decubitus position, the arm elevated away from the body, and the Upper Extremity Positioner lying on a table, in a position prior to adjustment to receive the arm.

FIG. 18. A patient lying in a right lateral decubitus position with the left arm positioned on the upper extremity positioner in preparation for a procedure

### DESCRIPTION OF THE PREFERRED EMBODIMENTS

Before explaining the disclosed embodiments of the present invention in detail it is to be understood that the invention is not limited in its application to the details of the particular arrangements shown since the invention is capable of other embodiments.
40 Restraining tie
50 Base plate (distal end)
51 Base plate (proximal end)
60 Single arm lower arm of hinge
70 Locking pin (for hinge)
75 Lower arm hole (for locking pin)
80 Proximal attachment plate for lower arm of hinge
81 Rivet for attaching proximal attachment plate to single arm lower hinge
90 Distal attachment plate for lower arm hinge
100 Sliding clasp
105 Rivet attachment for sliding clasp
110 Transitioning hinge arm
120 Rivet attachment for transitioning hinge arm to double arm upper hinge
130 Double arm upper arm of hinge
135 Upper Arm mounting tabs
140 Rivet attachment for hinge arms
150 Pivot holes (in mount for elbow plate)
160 Mount plate for proximal platform
170 Proximal platform
171 Proximal platform screw
172 Hole for proximal platform screw
173 Hole in mount for proximal platform
174 Washer for elbow proximal platform
200 Hole in proximal platform for restraining tape
210 Pillar support assembly
230 Pillar
240 Socket
245 Notch in socket
250 Locking mechanism for ball (in socket)
260 Distal platform
265 Hole in distal platform for ball and socket assembly
270 Ball and Stem assembly
275 Washer (for ball and stem assembly )
280 Hole for connecting pins
290 Notch in distal platform for restraining tape
300 slot for restraining tape
340 Intercalating board
350 Connecting Pins

### DETAILED DESCRIPTION OF THE FIGURES

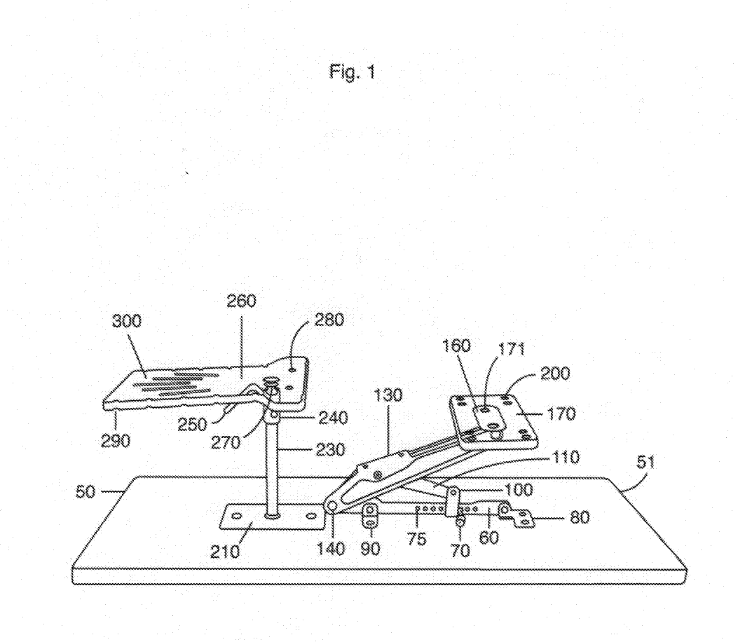

FIG. 1. The Upper Extremity Positioner at a side view. The distal end is to the left of the picture. The distal platform (260) lies in a neutral position relative to the plane of the base plate (50,51). The base plate is constructed of sturdy materials sufficient to support the weight of its attachments (as shown) and of a human upper extremity. The distal platform (260) attaches to the pillar (130) by a ball, stem and socket assembly (240, 270) and the pillar (130) attaches to the support board (50) by a pillar support assembly. A hinge mechanism with an upper arm (130) and a lower arm (60) attach to the support board by proximal (80) and distal (90) mounts. The upper arm (130) is shown in an elevated position. The proximal platform (170) lies us a neutral position parallel to the support board (51).

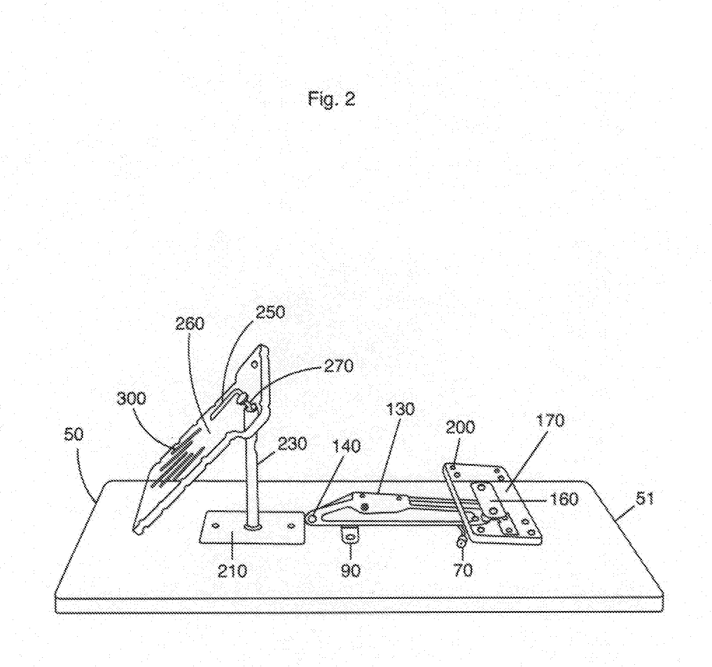

FIG. 2. The Upper Extremity Positioner is placed at a side view with the distal platform (260) in a tilted position. Functions of the ball-and-socket joint during flexion (distal end of the distal platform downward towards the base plate) necessarily cause some long axis (ie referring to long axis of the base plate) translation as shown by the more distal position of the proximal side of the distal platform. The wide range of flexion is demonstrated by the distal platform (260) with the ball and stem (270) attached in a preferred position at the proximal one-third of the distal platform (260) allowing a near complete range of motion for the wrist and hand. The proximal platform (170) lies in a neutral position parallel with the support board (51). The proximal platform (170) is shown fully reclined; the upper arm (130) of the working hinge lies parallel to and partially covers the lower arm of the hinge.

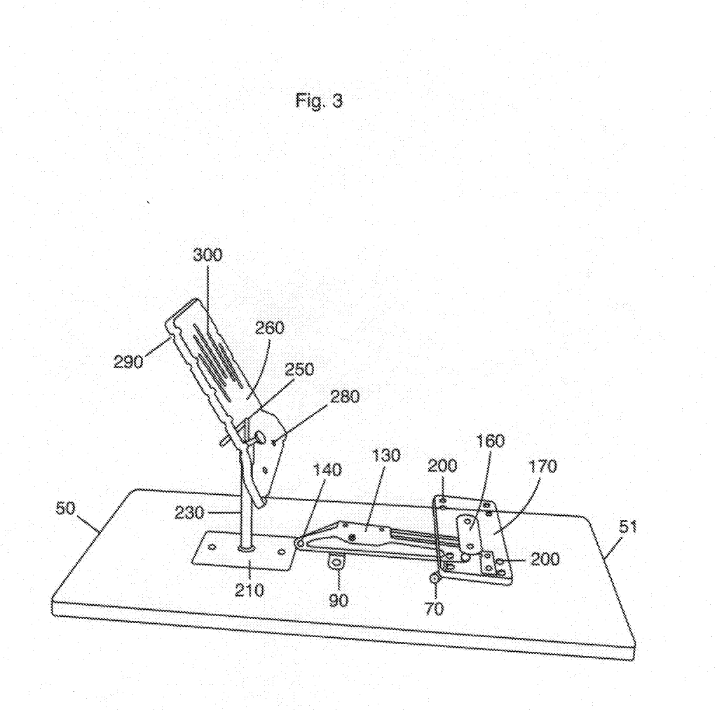

FIG. 3. The Upper Extremity Positioner is placed at a side view with the distal platform in an upwardly tilted position and the proximal platform fully reclined. The wide range of possible positions is further demonstrated and implied by the degree of extension (ie distal end of the distal platform deflected upward). The distal platform (260) has its distal end in an elevated position with the proximal platform in a fully reclined and otherwise neutral position. The position of the ball and stem (270) within the proximal one-third of the distal platform allows the distal end of the distal platform to be elevated to a nearly vertical position.

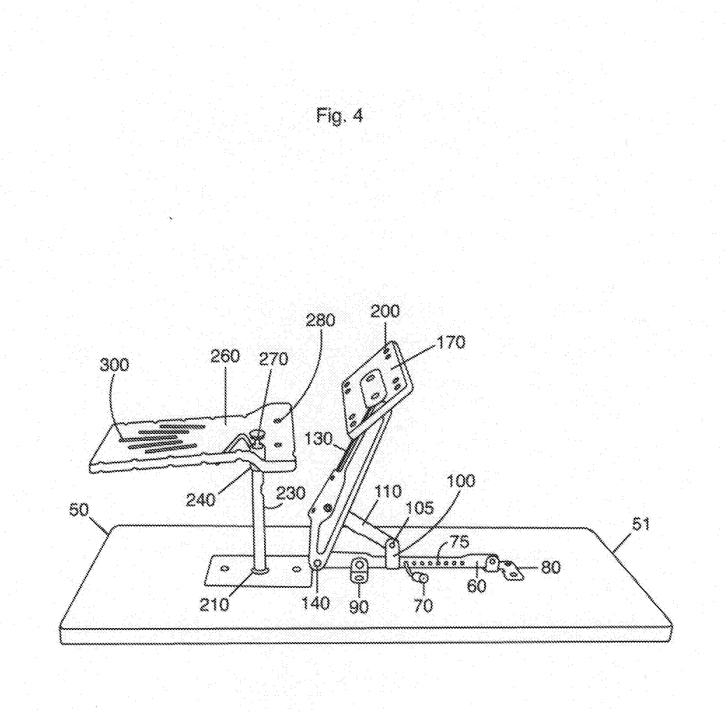

FIG. 4. The Upper Extremity Positioner is placed at a side view with the distal platform in a neutral position and the upper arm (130) of the hinge in near full elevation with the proximal platform in a tilted position. The wide range of available positions for the portion of an arm lying proximal to the wrist is demonstrated and otherwise implied by the elevated upper arm (130) and by the distal platform (260) that lies in a maximally forward tilted position. The locking pin (70) maintains the position of the arm by presenting the sliding clasp (100) from moving proximally, along the base plate, by the weight of a patients arm (upper extremity).

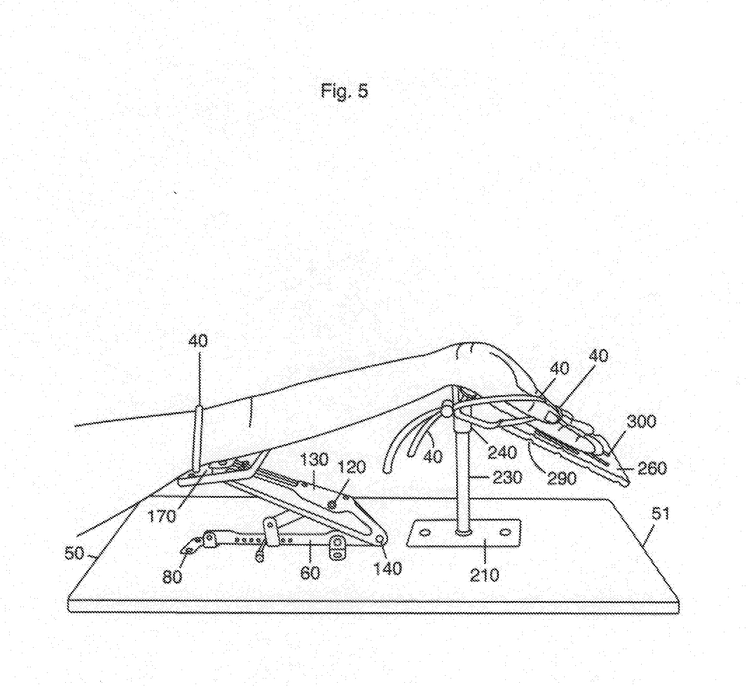

FIG. 5. The upper extremity positioner is viewed holding an upper extremity in a surgical position. Restraining ties (40) hold a portion of the arm proximal to the elbow at the proximal platform and portions of the hand to the Upper Extremity Positioner at the distal platform (260). The wrist is positioned in flexion and the proximal platform (170) accommodates and holds a comfortable and utilitarian position of the elbow as well as the entirety of the upper extremity proximal to the wrist.

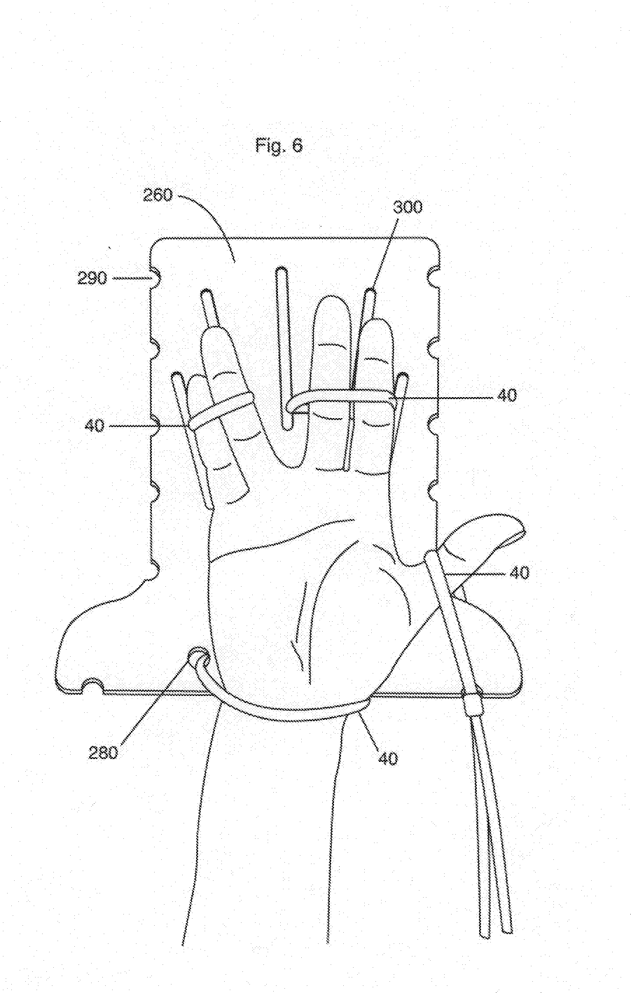

FIG. 6. The distal platform holding a hand and wrist by restraining ties. Slots at the interior of the distal platform, for the restraining ties (300) allow selective restraint of one or all of the digits. Holes (280) for the restraining ties at the proximal platform give the option for restraining the wrist (40, restraining tie) as might be done for a procedure on a finger. Notches (290) are indentations at the periphery of the distal platform and can also be used to secure a restraining tie (40) as demonstrated for the thumb in this drawing. Receiving holes for connecting pins (280) can also be used to pass restraining ties to secure the wrist.

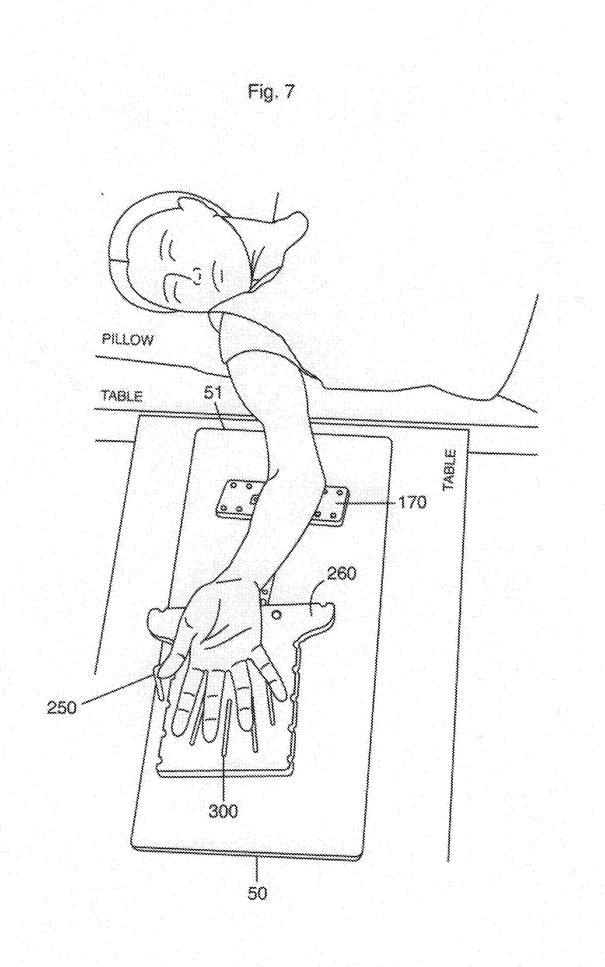

FIG 7. A patient in a supine position with the upper extremity lying upon the Upper Extremity Positioner which has been placed on a table. A position of the entire upper extremity upon the upper extremity positioner apparatus is shown in this picture. This view teaches a “three point” upper extremity support principle with support for the arm in this supine position by 1) the shoulder as the most proximal support and then by 2) the proximal platform and 3) the distal platform. The embodiment shown requires that the table be used to support the apparatus whilst a patient lies on a standard patient support appliance.

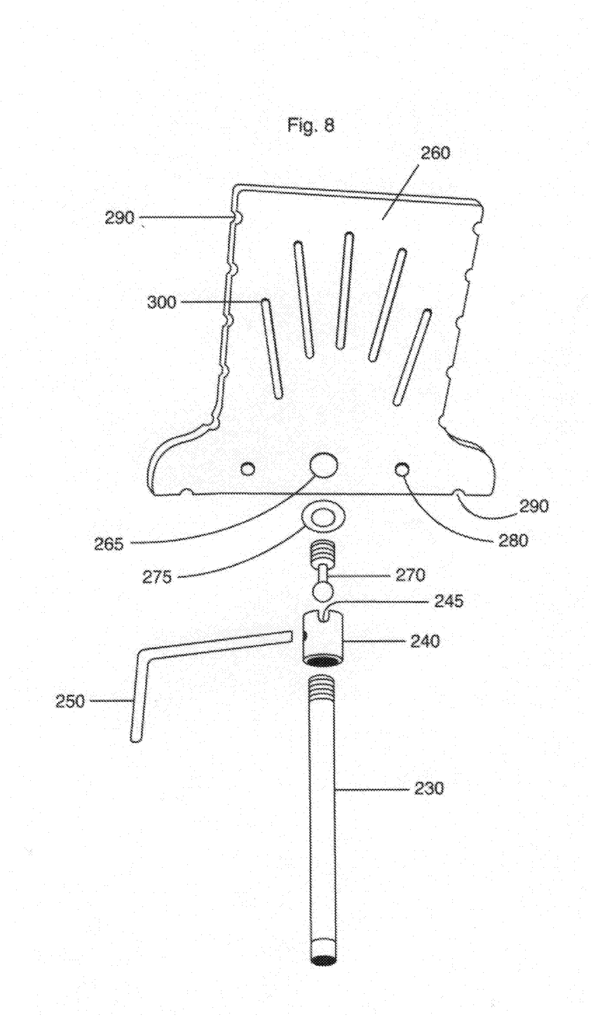

FIG. 8. Exploded view of the distal platform and its attachments to the pillar without the pillar support assembly. The view clearly shows the universal joint mechanism, (270 and 240, the ball-and-socket assembly) eccentrically placed toward an edge of the table in a preferred position for the purpose of producing a rotation of axis for the upper plate as close to the same edge as possible. The distal platform is flat, smooth and is suitable for hand-wrist contact and support. The edges of the distal platform are lined with notches (290) that serve to hold digital restraint ties (40). The proximal end of the distal platform (260) in this embodiment has a conspicuous expansion of the proximal end designed for a right or left thumb to be anchored by digital restraint ties (40) that can be anchored through notches (290) at the periphery of the platform. Several small holes (280) are the receiving holes for the connecting pins of the intercalating platform or for restraint ties (40) so that the wrist can be secured to the plate in this way, if needed. The distal platform (260) contains an opening (265) near an edge through which a threaded washer (275) is placed. A ball-and-stem (270) assembly screw into the threaded washer, specifically, by the threaded portion of the stem. The ball fits into the socket (240), that attaches to a pillar. The socket can be notched (240) for the purpose of allowing the stem, when necessary, to gain additional motion in a desired plane with a maximum of 90 degrees of motion in any plane. The socket is threaded and receives the pillar (130). Rotation of the plate is theoretically infinite and (240) can be rotated to the desired position by placing the notch at the desired location. The locking mechanism (250) is a modified “L” shaped screw that increases pressure against the ball within the socket by clockwise rotation.

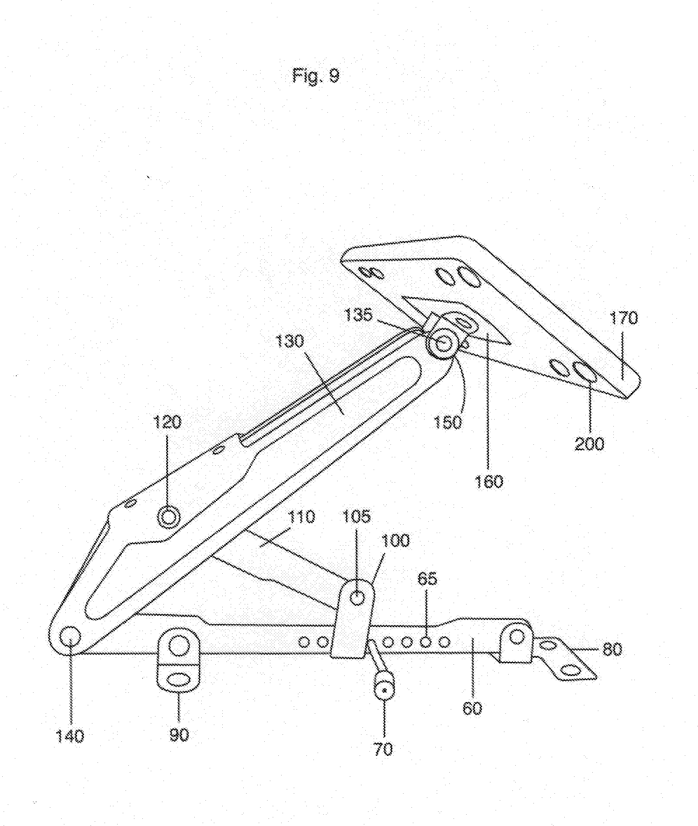

FIG. 9. The hinge assembly is shown with the upper arm in art elevated position and the proximal platform in a tilted position. This view is an enlarged view of the hinge which is created by joining (140, rivet) an upper arm (130) to a lower arm (60) (60,140,130). The locking pin (70) prevents the sliding clasp (100) from moving proximally under the weight of an arm. The proximal platform (170) moves freely by a mounted attachment to the upper arm.

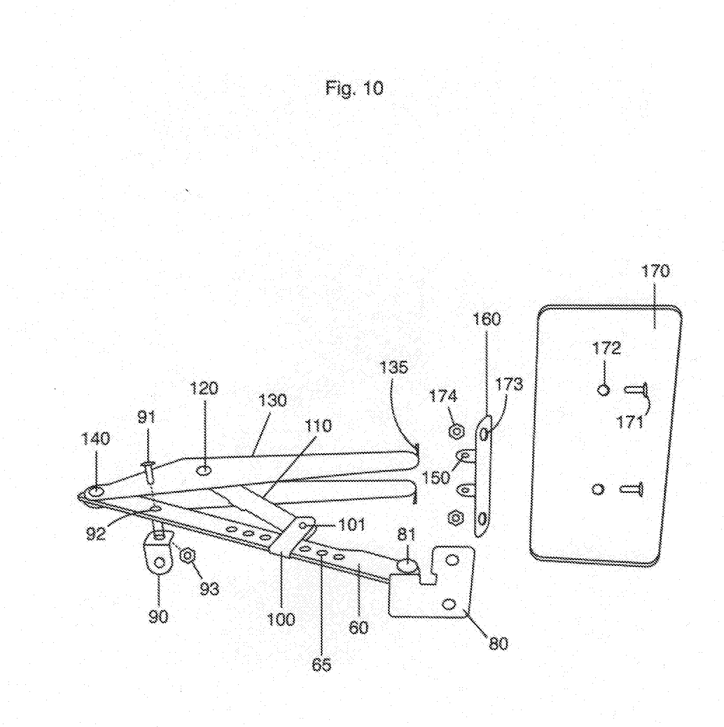

FIG. 10. Exploded view of the (dual) hinge assembly. The proximal platform (170) attaches to the mount for the platform (160) by a pair of screws and washers. The mount for the proximal platform has a pair of extensions with holes (150, pivot holes) for the upper arm mounting tabs (135). The ability of the proximal platform (170) to rotate is restricted only by the size of the plate, the position of the plate relative to the base support (51) and the degree of elevation of the upper arm. The otherwise freely rotating proximal platform (170) accommodates any position of that portion of an arm proximal to the wrist, once the wrist has been placed against the hand plate (260). The upper arm is joined to the lower arm by a rivet (140) and also by a transitioning arm (110) that moves a sliding clasp (100) along the lower arm (60) as the hinge opens and closes. The lower arm is attached to a support board by screw mounts placed proximally (80) and distally (90).

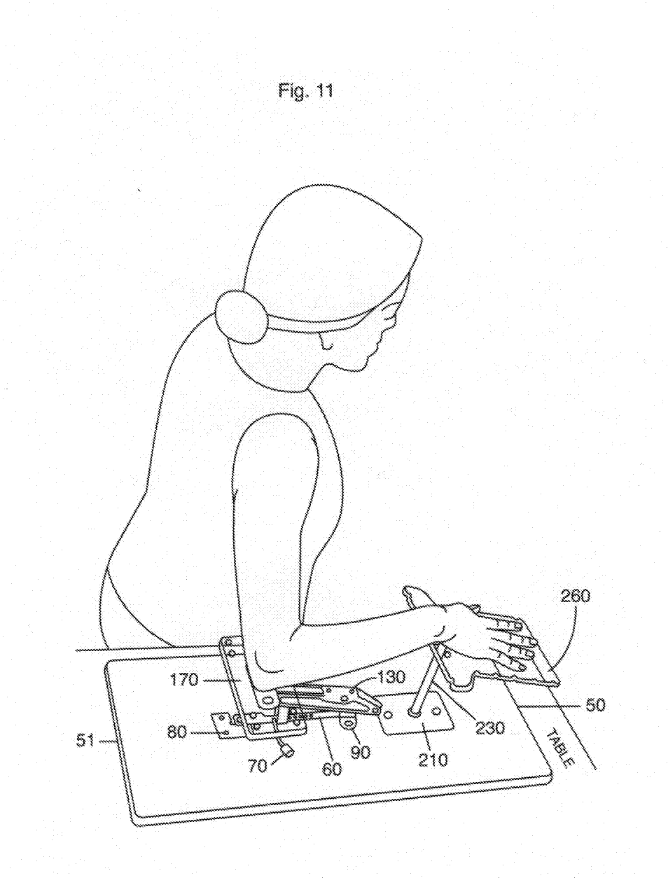

FIG. 11. A patient sitting in a chair with the right arm lying on the Upper Extremity Positioner depicts the versatility in positioning opportunities of the entire patient relative to the apparatus. The apparatus rests upon a table.

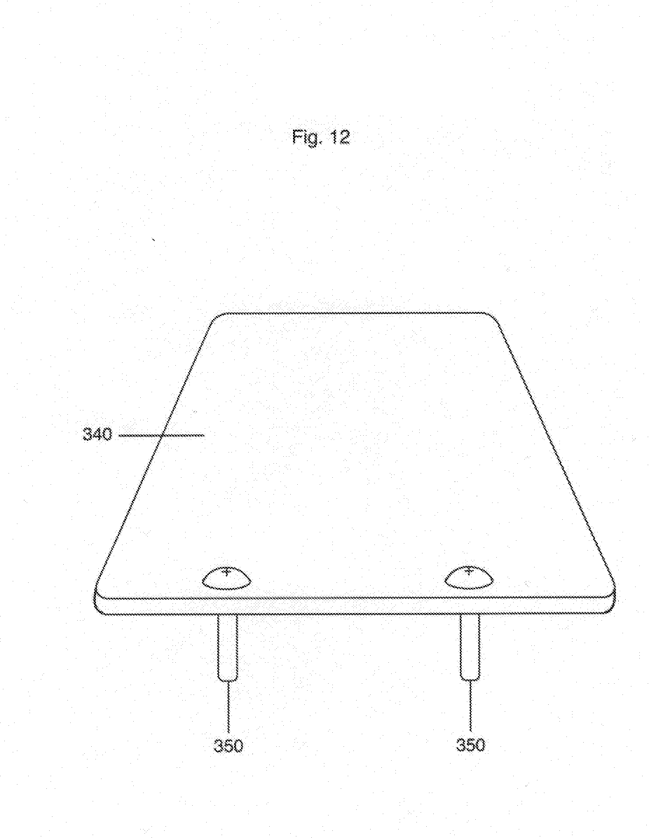

FIG. 12. The intercalating board (340) with connecting pins (350). The intercalating board is made to span the distance between the distal platform (260) and the proximal platform (170). The embodiment as shown is a flat shape constructed from a substantially supportive material not to exclude wood, glass, plastic, or metal. The pins are measured to match the distance between the receiving portion of the proximal hand plate. Drill holes are made in the intercalating plate and the pins are press fitted into position with an option of using an adhesive for additional binding strength.

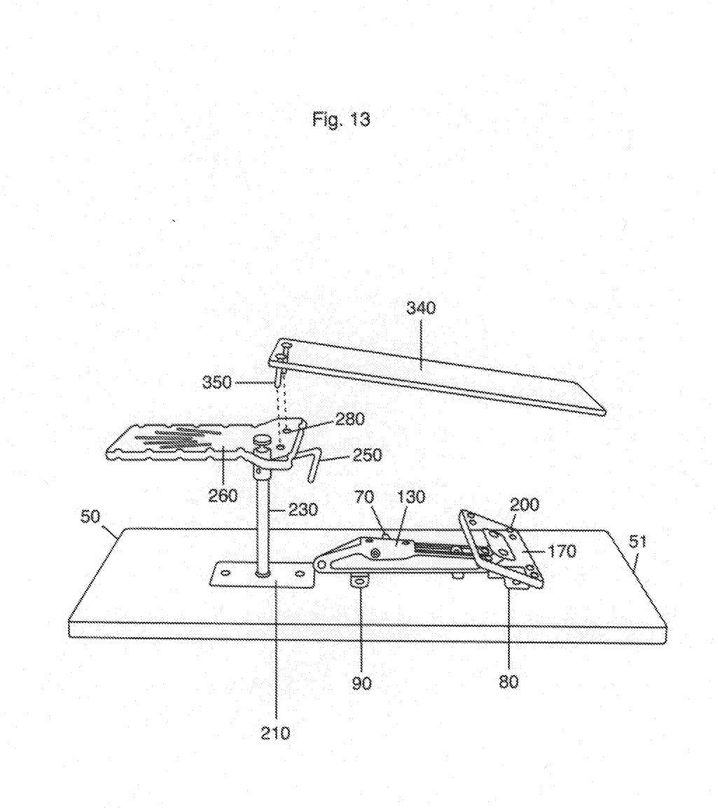

FIG. 13 Exploded view of the intercalating board positioned so that the connecting pins align with holes at the proximal end of the distal platform. The intercalating board is made to span the distance between the distal platform (260) and the proximal platform (170). The moveable parts of the Upper Extremity Positioner allow planar alignment of the proximal and the distal platforms such that a flat or contoured intercalating piece can be placed. The intercalating board (340) is placed over the distal platform (260) and the connecting pins are placed through the holes for the connecting pins (280). The proximal platform (170) is initially placed in a lowered position as shown so that the intercalating board (340) can be placed against the distal platform (260) without obstruction. As a final adjustment the proximal platform would be lifted so that it provides support for the intercalating plate and an upper extremity.

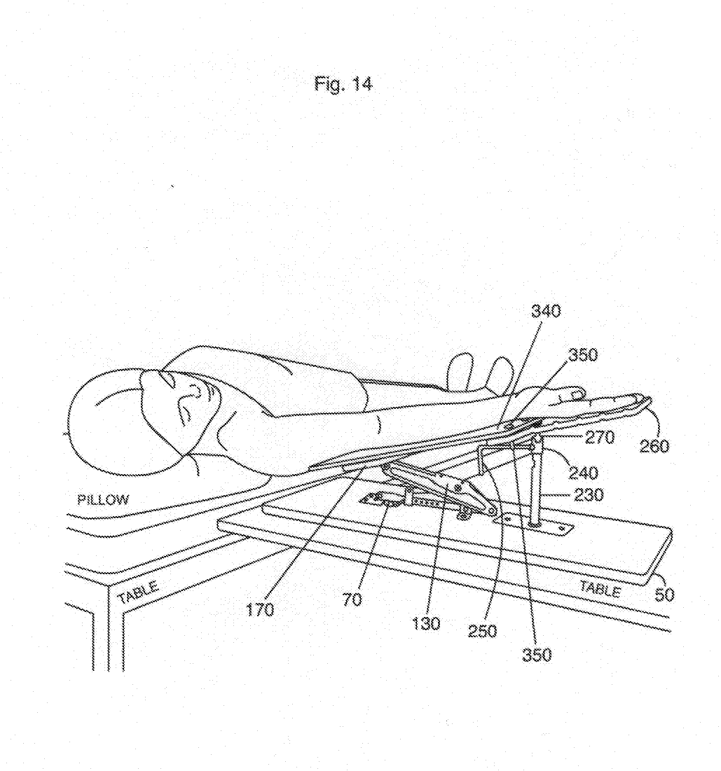

FIG. 14 A patient in a supine position with the arm held at a predetermined angle by the Upper Extremity Positioner with the intercalating board in place. The desired angle of the upper extremity can be accommodated by adjustments in the distal platform (260) and the proximal platform (170) with the flat intercalating (340) plate placed in between. In this embodiment the intercalating plate (340) rests passively upon the proximal platform (170) while being joined to the distal platform (260). In this way, a generous portion of the upper extremity is supported by a flat support surface as would be found with a traditional upper extremity board. Adjustments of the angle of The Upper Extremity Positioner with the intercalating board can easily be made by uniplanar adjustments of the distal platform and the proximal platform.

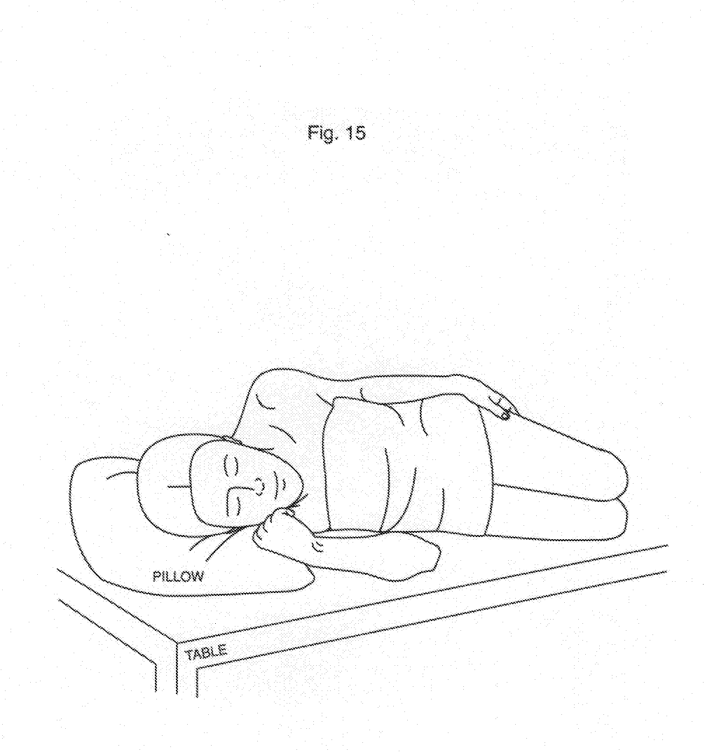

FIG. 15 A conscious patient lying in a right lateral decubitus position in preparation for positioning of the left arm on the Upper Extremity positioner.

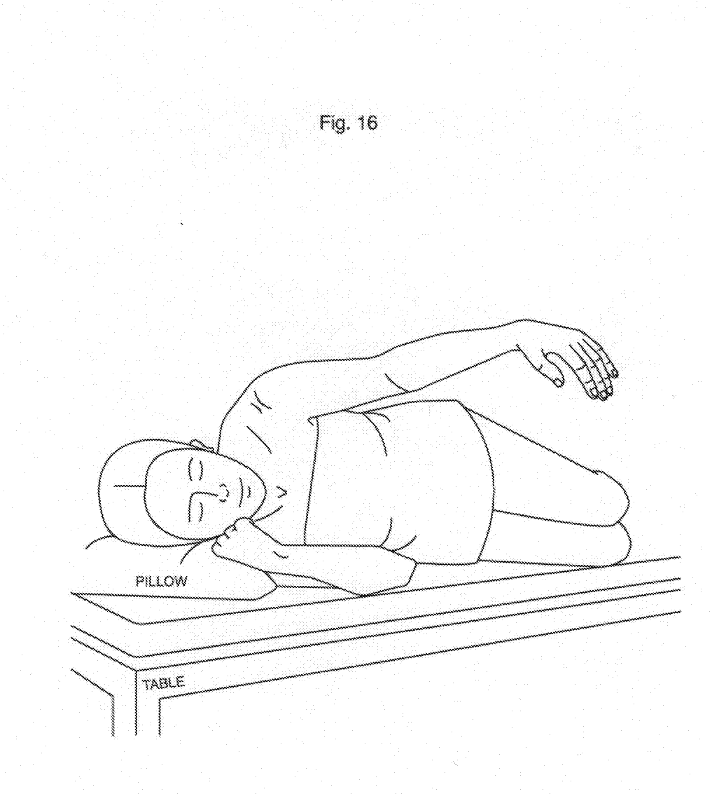

FIG. 16 A conscious patient lying in a right lateral decubitus position with the arm elevated away from the body. The patient is enacting a preferred position of the left upper extremity which can be held in the position by the cooperative patient or at assistant.

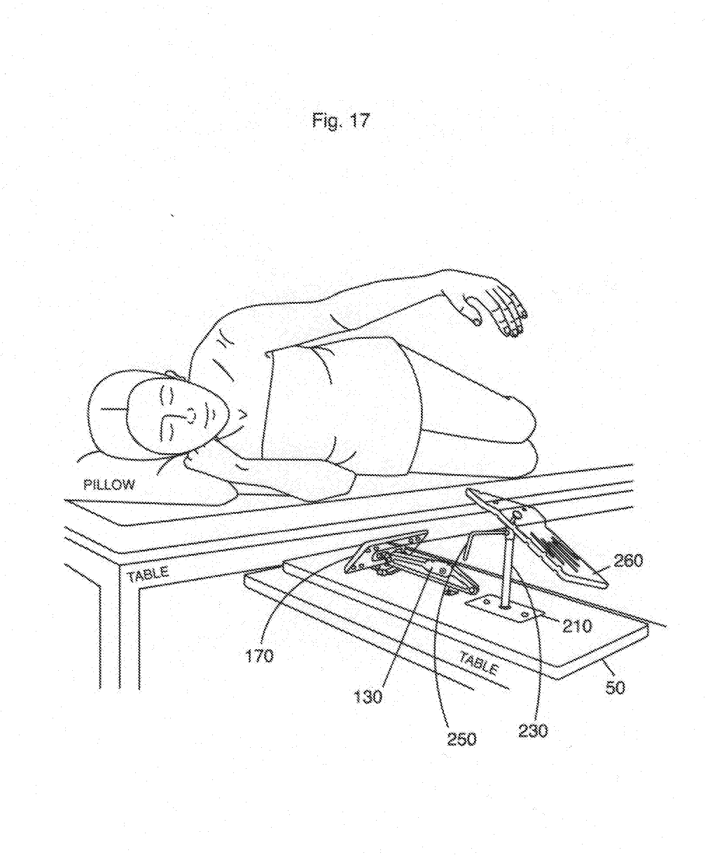

FIG. 17. A conscious patient lying in a right lateral decubitus position, the arm elevated away from the body, and the Upper Extremity Positioner lying on a table in a position prior to adjustment to receive the arm. The proximal platform (170) is in a lowered position and the distal platform (260) is adjusted to match the position of the postured hand and wrist of the patient.

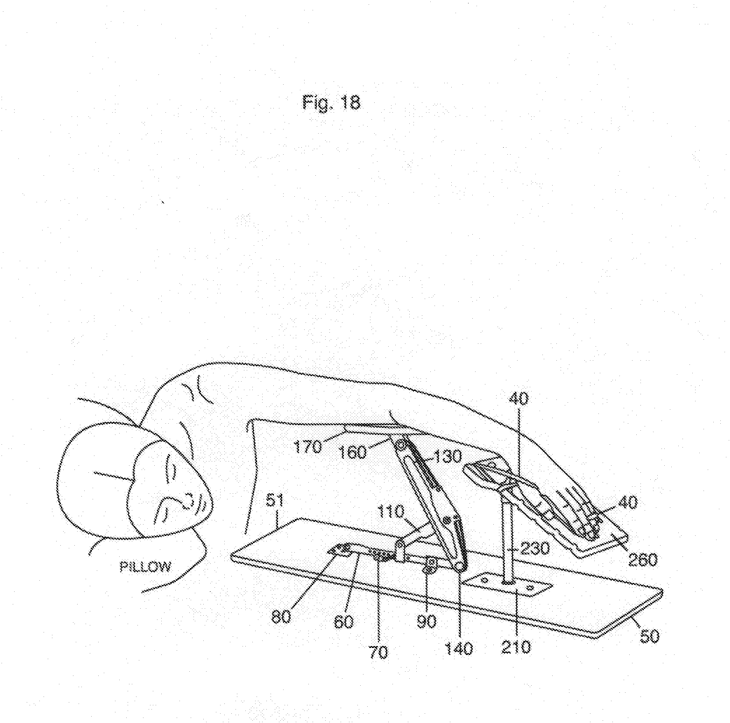

FIG. 18. A patient lying in a right lateral decubitus position with the left arm positioned on the upper extremity positioner in preparation for a procedure. The arm has been lowered to lie against the apparatus with the hand and wrist against the distal platform (260) and a portion of the arm proximal to the wrist lying against the proximal platform (170). The proximal platform has been elevated to support the position of the arm in a desired position.

### GLOSSARY

Above—synonym is “higher than”; see definition “Motion” relates to a position of a named element (of the presented embodiment of the Upper Extremity Positioner) with respect to another named element.

Anatomy—general term for any portion of an upper extremity including connecting portions of the shoulder girdle (see shoulder, shoulder girdle)

Arm board—synonymous with “arm board configuration”; a term given to an upper extremity support apparatus providing a substantially planar deck designed to provide surface beneath a portion of an extremity proximal to the elbow and also including the elbow, forearm, wrist and hand; An example of this type of device is shown in U.S. Pat. No. 6,336,412-B2.

Axis line—a line drawn to illustrate or define an axis of rotation

Base Plate—a substantially planar support surface for attachments of pillars, hinges, fasteners and other devices that aid in supporting an upper extremity.

Depression of the Upper arm—movement of the upper arm of the pivot hinge of the proximal platform toward a parallel position of the upper arm and the lower arm. it is important to note that the hinge as a part of the apparatus can only achieve such a position by movement in one direction (see FIG. 2).

Detent—structure of a machine that purposefully prevents motion of a part until the part is released

Distal—1. toward the finger tips when describing anatomy of the upper extremity; 2. towards the distal end of the apparatus as described in FIG. 1

Distal Elevated Platform—distal platform; that portion of the presented embodiment of the upper extremity positioner that holds the wrist and hand.

Elevated platforms—provides a perch for supporting an upper extremity by a pillar, column hinge, fastener, or other device aiding in the support of an extremity and coupled to the base plate. Each elevated platforms is considered to be independently moveable unless otherwise specified. The elevated platforms of the presented embodiment of the upper extremity positioner (ie, the distal elevated platform and the proximal elevated platform) are independently moveable and otherwise unconnected in space. A proximal elevated platform may have more elevation from the base plate than a distal elevated platform.

Enact—act out as if in a role or play on stage. Refers to a method step where the upper extremity is positioned “in the air” so that a surgeon or other practitioner can visualize the ideal serviceable position.

Hand—Anatomy distal to and including the carpometacarpal joints, not to exclude the digits

Height Differential (between the proximal and distal elevated platforms)—refers to the relative vertical displacements of the platforms, however, since both platforms are also pivotal there can be ambiguity about which portion of the platform is being referenced. When this type of ambiguity occurs the default reference point would be the most proximal nonpivotal element which for the proximal elevated platform (in the depicted embodiment) would be the mounting tabs of the upper arm of the hinge and for the distal elevated platform (in the depicted embodiment) would be the midpoint of the ball.

Long axis (of the Base Plate)—refers to the rectangular shape of the base plate as shown in the preferred embodiment (FIGS. 1-5,7,11,13-14,17-18) indicating the base plate shape has two equal length short sides and two equal length long sides. The long axis of this plate is created by drawing a line on the plate parallel to one of the long sides or by drawing a line on the plate perpendicular to one of the short sides.

Longitudinal Movement—movement of a support platform, hinge or other coupling device along the long axis line

Motion—also see “Upper”—1. Vertical Motion—synonym is elevational motion; motion in the “ground to sky” plane (while on Earth) or referring to motion of a hinge, platform or anatomic site “closer versus farther” from the base plate, where vertical movement would be farther from the base plate.

Perpendicular Rotational Axis—circular rotation of an object substantially around an axis line having a constant radius from the axis line; an example is the contact (street) surface of a tire rotating perpendicularly around the axle of the tire.

Pillar—the most generic term for any load hearing structure that serves to sustain elevation of a platform above the base plate; the term as applied to the presented embodiment of the upper extremity positioner refers specifically to the structure that sustains elevation of the distal elevated platform

Platform—any load bearing structure (other than the base plate) designed to contact anatomy

Proximal—1. towards the shoulder when describing anatomy of the upper extremity; 2. towards the proximal end of the apparatus as described in FIG. 1

Proximal Elevated Platform—proximal platform; that portion of the presented embodiment of the upper extremity positioner that holds anatomy proximal to the distal elevated platform

Restraining Tie(s)—a ligature or other rope like binding used for securing anatomy to the UEP

Serviceable—useable (as can be related to a given profession)

Short Axis (of the Base Plate)—refers to the rectangular shape of the base plate as shown in the preferred embodiment (FIGS. 1-5,7,11,13-14,7-18) indicating the base plate shape has two equal length short sides and two equal length long sides. The short axis of this plate is created by drawing a line on the plate parallel to one of the short sides or by drawing a line on the plate perpendicular to one of the long sides.

Shoulder (shoulder girdle)—synonym shoulder girdle. In the field of orthopedics the shoulder is generally considered to be the glenohumeral joint, the acromioclaviculr joint and their “nearby” (otherwise undefined precisely in literature) attachments. The “shoulder girdle” concept more fully embraces the all of the attachments “relevant” to shoulder function not to exclude, for example, the pectoralis major. The pectoralis major insertion along the proximal humerus could be considered a landmark for the proximal platform of the upper extremity positioner apparatus.

Universal joint—general term used for a mechanism providing at least two planes of motion for another device or tool component

Upper—synonymous with “higher” in the context of the “vertical motion” (see “Motion”) definition ie, the “nearer to the sky” (while on Earth) and the “farther from the base plate” of a hinge part, platform or anatomic site.

Vertical motion—see “Motion”

Wrist—the segment along the axial plane of the upper extremity between the more proximal of the distal radius and ulna articular surface and the carpometacarpal joint

I claim:

1. An apparatus for holding serviceable positions of an upper extremity, comprising;
a) a base plate with sufficient strength to support the weight of a plurality of platforms and the weight of an upper extremity,
b) a plurality of platforms with a minimum of;
1) a distal elevated platform for receiving a hand and a wrist coupled to the base plate by a pillar, and
2) a proximal elevated platform coupled to the base plate by means to adjustably receive anatomy proximal to the distal elevated platform.

2. Distal elevated platform of claim 1 wherein restraining ties can secure the hand and wrist at the periphery (of the distal elevated platform) by an array of notches, and through the interior by a series of slots.

3. Distal elevated platform of claim 2 further including a pair of holes for connecting pins that receive the connecting pins of an intercalating platform.

4. Pillar of claim 1 further including a universal joint providing means for the distal elevated platform to be moved through a multiplicity of spatial positions.

5. Universal joint of claim 4 wherein a ball and socket mechanism directs motion of the distal elevated platform.

6. Ball and socket mechanism of claim 5 further providing long axis displacement of the distal elevated platform relative to the base plate.

7. Ball and socket mechanism of claim 6 wherein a detent mechanism, threadably coupled to the pillar, releasably prevents motion of the ball within the socket thereby securing a desired angle of the distal elevated platform relative to the base plate.

8. Upper extremity table of claim 1 wherein a hinge means provides vertical and pivotal motion to the proximal elevated platform.

9. Hinge means of claim 8 wherein a first hinge lies proximal to the pillar and has an upper bar and a lower bar coupled at a single pivot point providing vertical and long axis movement of the proximal end of the upper bar with respect to the base plate, and couples to the base plate by the lower bar which aligns with the long axis of the base plate.

10. Hinge means of claim 9 further including a second hinge that couples the upper arm of the first hinge to the proximal elevated plate providing means for rotation for the proximal elevated plate with respect to the base plate.

11. Means for rotation of claim 10 further including a mount plate for the proximal platform which couples the proximal elevated platform to the upper arm of the first hinge by having pivot holes that encircle upper arm mounting tabs that rotate within the confines of said pivot holes thereby guiding the proximal elevated plate in a substantially perpendicular direction with respect to an axis line drawn from the center of one pivot hole to another pivot hole.

12. First hinge of claim 9 wherein a locking pin can engage the lower arm to prevent motion of a sliding clasp along said lower arm which in-turn holds the upper arm at a predetermined position.

13. Intercalating platform of claim 3 wherein means for bridging the space between the proximal elevated platform and the distal elevated perform can be implemented when the surface of said proximal elevated platform lies substantially in the same plane (uniplanar) as the surface of said distal elevated platform.

14. Means for bridging the space between the proximal elevated platform and the distal elevated platform of claim 13 wherein elongated peg-like members extending from a first end of said intercalating platform engages the distal elevated platform through receiving holes until the intercalating platform lies substantially flush against said distal elevated platform, whereupon the proximal elevated platform can then be vertically and rotationally adjusted to contact and lie flush against the undersurface of a second end of the intercalating support segment thus producing an arm board configuration.

15. An adjustable arm board for supporting a human upper extremity, comprising;
a) a base support that may be placed on a table or other suitable supportive surface,
b) a distal elevated platform;
(1) having suitable form for receiving and holding the baud and the wrist,
(2) coupled to the base plate by a pillar, and
(3) having a universal joint that tiltably adjusts said distal elevated platform relative to the base plate,
c) a proximal elevated platform coupled to the base support by means for elevational, longitudinal, and rotational movement of the proximal elevated platform relative to the base support to adjustably receive anatomy between the shoulder and the wrist, and
d) an intercalating platform with means to provide a bridging surface between the distal elevated platform and the proximal elevated platform.

16. Intercalating platform with means to provide a bridging surface of claim 15 wherein a supportive surface having a length, a width and a thickness is formed from stout material that integrates peg-like extensions that cat couple to an elevated platform.

17. Elevated platform of claim 16 wherein holes are placed at the interior of the distal elevated platform to receive peg-like extensions from the intercalating plate at a predetermined angle.

18. Predetermined angle of claim 17 wherein the intercalating platform is supported by a portion of the distal elevated platform that lies substantially uniplanar to a portion of the proximal elevated platform, with the angle of the intercalating platform, relative to the base plate, determined by the height differential between the distal elevated platform and the proximal elevated platform.

19. Means for elevations, longitudinal and rotational movement of the proximal elevated platform relative to the base support of claim 15 wherein a series of hinges coordinate motion of said proximal elevated platform; a first hinge is an adjustable pivot hinge providing vertical and longitudinal movement of the proximal end of the upper bar with respect to the plane of the base plate, by an upper bar and a lower bar, with said lower bar coupled to the base plate proximal to the pillar and aligned along the longitudinal axis of the base plate, and the pivoting end of said first hinge positioned closest to the pillar, wherein a locking pin can engage the lower arm and prevent relative motion between the lower arm and the upper arm at a predetermined position by halting the movement of a transitioning hinge arm; and a second hinge that couples the upper arm of the first hinge to the proximal elevated plate, providing rotational motion to the proximal elevated plate in a substantially perpendicular plane to the short axis of the base plate with said second hinge further including a mounting bracket for the proximal elevated platform with receiving holes that allow the contoured extensions of the upper arm to pivot within the confines of the receiving holes.

20. A method for performing upper extremity procedures so as to facilitate ready access of the anatomy thereto by a surgeon or other practitioner, comprising:
Placing the patient in a desired lying, sitting or standing orientation,
Positioning the upper extremity by enacting the ideal serviceable alignment,
Utilizing a table for positioning the upper extremity having;
a base support that may be supported on a surgical table or other suitable support,
a distal elevated platform, with suitable form for receiving the hand and the wrist, mounted on a pillar attached to the base support, said pillar having means for tiltable adjustment of the distal elevated platform relative to the base plate,
a proximal elevated platform coupled to the base support to adjustably receive anatomy between the shoulder and the wrist, having means for elevation, longitudinal and parallel rotational movement relative to the base support.
Placing the table for positioning the upper extremity on a support surface,
Moving the table for positioning the upper extremity beneath the positioned extremity,
Adjusting the distal elevated platform hand to achieve proper length relationships of the arm and the apparatus,
Lifting the proximal elevated platform toward anatomy proximal to the wrist,
Applying restraining ties as needed to secure the upper extremity to the proximal elevated, platform and to the distal elevated platform, whereby an upper extremity lies in a serviceable position to begin a procedure.
